(19) United States
(12) Patent Application Publication
(10) Pub. No.: US 2022/0096128 A1
(43) Pub. Date: Mar. 31, 2022

(54) ILIAC CREST DISPLACEMENT DEVICE AND METHOD

(71) Applicant: NuVasive, Inc., San Diego, CA (US)

(72) Inventor: Juan Uribe, Tampa, FL (US)

(21) Appl. No.: 17/484,304

(22) Filed: Sep. 24, 2021

Related U.S. Application Data
(60) Provisional application No. 63/083,711, filed on Sep. 25, 2020.

(51) Int. Cl. A61B 17/70; A61B 17/88

(52) U.S. Cl. CPC A61B 17/7022; A61B 2017/564; A61B 17/8866; A61B 17/8869

(57) ABSTRACT

The disclosure relates to an iliac crest displacement device comprising: an engagement member for engaging an iliac crest of a subject; and a pulley assembly for causing the engagement member to move the iliac crest inferiorly, thereby displacing the iliac crest relative to a rib cage of the subject.

### TECHNICAL FIELD

The subject matter described herein relates to an iliac crest displacement device and methods associated therewith.

### BACKGROUND

A noteworthy trend in the medical community is the move away from performing surgery via traditional “open” techniques in favor of minimally invasive or minimal access techniques. Open surgical techniques are generally undesirable in that they typically require large incisions and high amounts of tissue displacement to gain access to the surgical target site, which produces concomitantly high amounts of pain, lengthened hospitalization (increasing health care costs), and high morbidity in the patient population. Less-invasive surgical techniques (including so-called “minimal access” and minimally invasive” techniques) are gaining favor due to the fact that they involve accessing the surgical target site via incisions of substantially smaller size with greatly reduced tissue displacement requirements. This, in turn, reduces the pain, morbidity and cost associated with such procedures. On such minimally invasive approach, a lateral trans-psoas approach to the spin, developed by NuVasive, Inc., San Diego, Calif. (XLIF®) has demonstrated great success in reducing patient morbidity, short length hospitalization and fast recovery time if it is employed. Even more recently, lateral techniques are being performed in the prone position.

FIG. 1 shows human anatomy 5 including a rib cage 10 having true ribs 12 and floating ribs 14, thoracic vertebrae 16, lumbar vertebrae 18, a pelvis 20, a coccyx 22, a sacrum 24, an ilium 26 and iliac crest 28. It is often difficult to access retroperitoneum (not shown in FIG. 1) and the thoracic 16 and lumbar spine 18 during surgery. Unfortunately, the iliac crest 28 blocks lateral access to the L5/S1 disc space in most individuals. In some individuals the height of the iliac crest 28 may extend upwards to a point where it also inhibits or challenges lateral access to the L4/L5 disc space. The systems and methods described herein are directed towards eliminating or at least reducing, these challenges.

### SUMMARY

A first aspect of the disclosure relates to an iliac crest displacement device comprising: an engagement member for engaging an iliac crest of a subject; and a pulley assembly for causing the engagement member to move the iliac crest inferiorly, thereby displacing the iliac crest relative to a rib cage of the subject.

The engagement member can include a hook, saddle, grasp, anchor, latch, or clamp. In some embodiments, the device further includes a pin configured to be fixed to the iliac crest, and the engagement member is configured to couple with the pin. The pin can include an anchor, a traction pin, or a fixation element.

The pulley mechanism can include a pulley and a tensioning mechanism configured to provide traction and/or tension to the pulley. The pulley can include a tie, cord, tether, strap, line, band, rope, or wire. A first end of the pulley is coupled to the engagement member. A second end of the pulley, opposite the first end, can be configured to be grasped by a medical professional to move the iliac crest inferiorly. A second end of the pulley, opposite the first end, can be configured to be affixed to a surgical table or bar.

The tensioning mechanism is configured to provide traction and/or tension to the pulley. The tensioning mechanism can include a ratchet tensioner.

In some embodiments, at least a portion of the pulley is configured to be positioned subcutaneously about the subject, and wherein a remainder portion of the pulley and the tensioning mechanism is configured to be positioned external to the subject.

A second aspect of the disclosure relates to a method for accessing a surgical target site of a subject. The method may include: accessing an iliac crest of a subject; providing an iliac crest displacement device, the iliac crest displacement device including: an engagement member for engaging the iliac crest of the subject; and a pulley assembly for causing the engagement member to move the iliac crest inferiorly; engaging the iliac crest with the engagement member; using the pulley assembly to cause the iliac crest to move inferiorly thereby displacing a pelvis of the subject relative to a rib cage of the subject; and providing tension to the pulley to hold the position of the iliac crest in the displaced position.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 shows a human anatomy;

FIG. 2 shows a device according to embodiments of the disclosure;

FIG. 3 shows a device according to embodiments of the disclosure;

FIG. 4 shows a device according to another embodiment of the disclosure; and

FIG. 5 shows a device according to embodiments of the disclosure being used with a subject in the prone position.

It is noted that the drawings of the subject matter are not necessarily to scale. The drawings are intended to depict only typical aspects of the subject matter, and therefore, should not be considered as limiting the scope of the disclosed subject matter. In the drawings, like numbering represents like elements between the drawings.

### DETAILED DESCRIPTION

The disclosure relates to a device and/or system 100 for facilitating displacement of the iliac crest/pelvis relative to the lumbosacral spine to increase a longitudinal distance between the rib cage and the pelvis allowing access to retroperitoneum and access to the thoracic and lumbar spine laterally, anteriorly, and/or posteriorly.

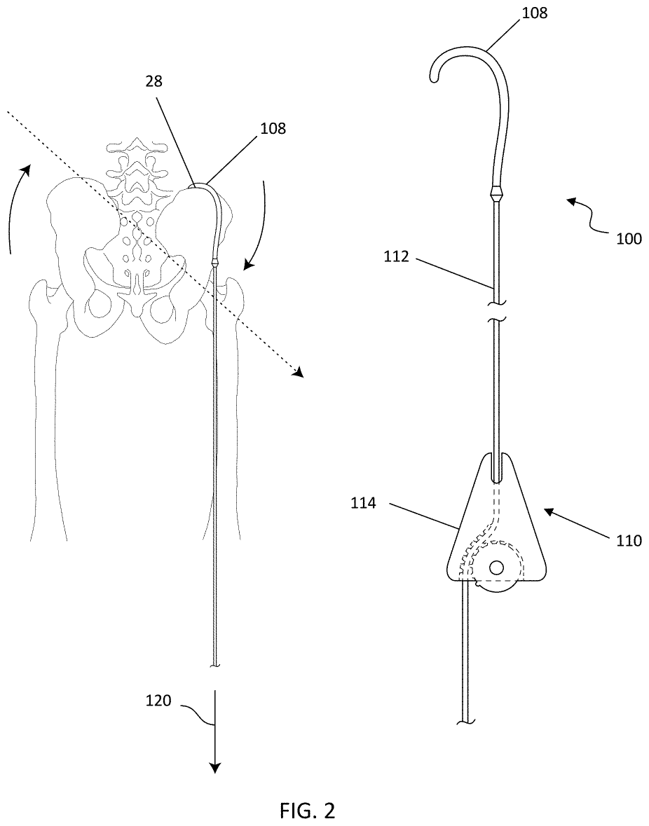

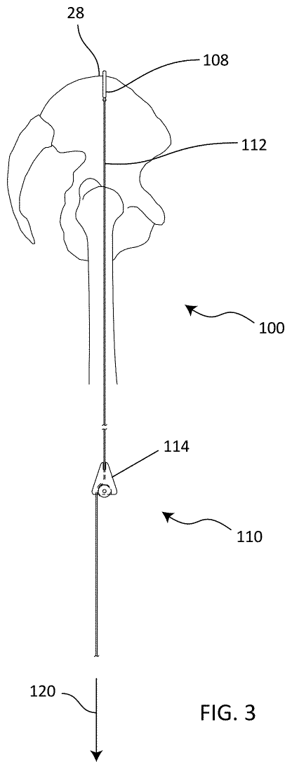

The device 100 includes an engagement member 108 for engaging the iliac crest 28. The engagement member 108 may include a hook, saddle, grasp, anchor, latch, clamp and/or any other now known or later developed means for engaging the iliac crest 28. The engagement member 108 is configured to engage, affix, grip and/or grab on to the iliac crest 28. In some embodiments, the engagement member 108 is bendable or deformable such that a user (e.g., a medical professional such as a surgeon or surgical assistant), can achieve a desired shape and/or geometry of the engagement member 108 in order to better engage, affix, grip and/or grab on to the iliac crest 28. In some embodiments, such as where the engagement member 108 includes a hook as shown in FIGS. 2-3, the engagement member 108 can at least partially engage opposing sides of the iliac crest 28 (e.g., anterior and posterior sides), as well as a superior surface to provide better support and engagement.

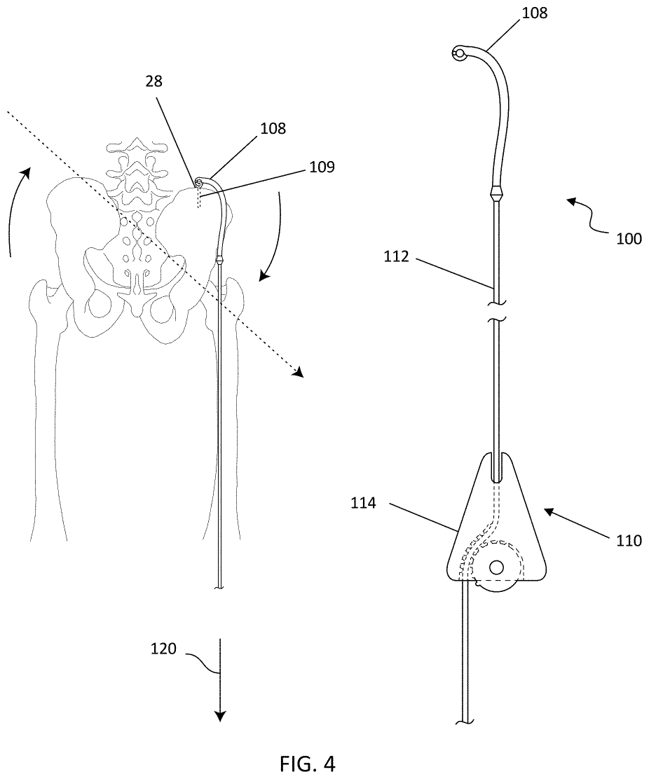

In other embodiments (FIG. 4), a pin 109 may be inserted within the iliac crest 28 and engaged by the engagement member 108. The pin 109 can include an anchor, traction pin, fixation element and/or any other now known or later developed fixation means that is capable of being fixed to the iliac crest 28. In some embodiments, at least a portion of the pin 109 includes threads for threading into the iliac crest 28. In such an embodiment, the engagement member 108 is configured to engage and/or couple to the pin 109 directly As such, the engagement member 108 according to this embodiment can include an end configured to couple and uncouple to the pin 109. In one example embodiment, the pin 109 can include a hook or ring at an end thereof for receiving a complementary hook or clasp of the engagement member 108. In other example embodiments, the pin 109 and engagement member 108 may each have a geometry at ends thereof configured to mate and couple together. The present disclosure contemplates any mating configuration of the pin 109 and engagement member 108 that ensures secure engagement of the pin 109 and engagement member 108 enabling displacement of the iliac crest 28.

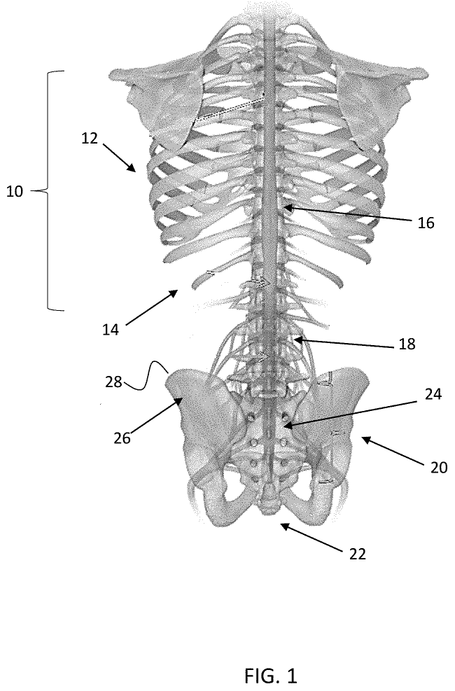

The device 100 may also include a pulley assembly 110 having a pulley 112 and an optional tensioning mechanism 114. The pulley 112 can include a tie, cord, tether, strap, line, band, rope, wire, and/or any other now known or later developed pulley means that is capable of being used with the pulley assembly 110. The pulley assembly 110 is configured to move the engagement member 108 in an inferior or caudal direction 120 thereby causing the iliac crest 28 to move away from the rib cage 10 (FIG. 1) and lumbosacral spine 18, 24 (FIG. 1). Specifically, one end of the pulley assembly 110 (e.g., the pulley 112) is attached, affixed, and/or fastened to the engagement member 108 and the other, free end opposite the engagement member 108 can be held by a medical professional and pulled or manipulated to cause movement of the iliac crest 28. In some embodiments, a handle or gripper may be removably attached to the free end for better handling of the pulley by the medical professional.

In some embodiments, the free end of the pulley 112 opposite the engagement member 108 can be attached, affixed, secured and/or fastened to a stationary object (e.g., a surgical table or bar) and the tensioning mechanism 114 can be used to provide traction and/or tension to the pulley 112. That is, where the tensioning mechanism 114 provides traction, the tensioning mechanism 114 can be actuated to provide additional movement of the pulley 112 thereby displacing the iliac crest 28, and where the tensioning mechanism 114 provides tension, the tensioning mechanism 114 can be actuated to pull tight and secure the pulley 112, thereby securing the displacement of the iliac crest 28. The tensioning mechanism 114 can include a ratchet tensioner, buckle, strap slip lock, and/or any other now known or later developed means capable of providing tensioning to the pulley 112 within the pulley assembly 110. Where a ratchet tensioner is used the tensioning mechanism 114 includes ratchet teeth for engaging with the pulley 112 providing traction and tension 112 thereto. In some embodiments, the tensioning mechanism 114 is motorized. The tensioning mechanism 114 may be operatively associated with the pulley 112 to provide traction and/or tension thereto. In some embodiments, the tensioning mechanism 114 can be removably associated to the pulley 112 about a length of the pulley 112.

The pulley assembly 110 can be actuated to pull the iliac crest 28 inferiorly/caudally and the tensioning mechanism 114 then provides traction and/or tension on the pulley 112 to hold the position of the iliac crest 28 while a desired surgical technique is performed. In some embodiments, at least a portion of the pulley 112 can be positioned subcutaneously and extend from the engagement member 108 in the inferior/caudal direction 120 subcutaneously while the remainder of the pulley 112 and the tensioning mechanism 114 are positioned external to the subject, i.e., supracutaneously.

The pulley assembly 110 can be configured to actuate in two directions. In a first direction, the pulley assembly 110 causes the engagement member 108 to pull the iliac crest 28 inferiorly/caudally thereby causing the iliac crest 28 and the pelvis 20 to displace inferiorly/caudally relative to the rib cage 10. Once the tensioning mechanism 114 is released, the pulley 112 can move in the opposite direction (i.e., the superior direction or cranially) to loosen the tension being applied to the iliac crest 28 to allow the pelvis 20 to return to a natural, resting state.

Aspects of the disclosure also include a method for accessing a surgical target site on the spine of a patient, e.g., at or below the L4/L5 intervertebral disc through a substantially lateral approach. The method can include accessing the iliac crest 28 of a subject, providing the iliac crest displacement device 100, engaging the iliac crest 28 with the engagement member 108, using the pulley assembly 110 to cause the iliac crest 28 to move inferiorly/caudally thereby displacing the pelvis 20 relative to the rib cage 10 of the subject. At this stage, the iliac crest 28 is positioned inferior/caudally to the target site. The method also includes, providing traction and/or tension to the pulley 112 to hold and/or maintain the position of the iliac crest 28 in the displaced position. At this point, the retroperitoneum and the thoracic 18 and lumbar spine can be accessed laterally, anteriorly, and/or posteriorly, and the desired surgical procedure and/or technique can be performed. Once complete, tension on the pulley 112 can be released allowing the pulley 112 to move in the opposite direction to loosen tension being applied to the iliac crest 28 to allow the pelvis 20 to return to a natural, resting state.

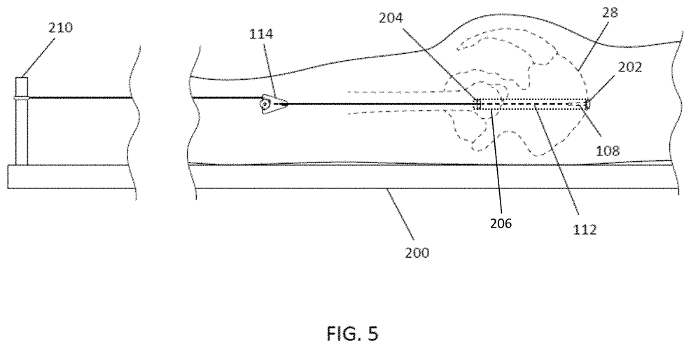

Referring to FIG. 5, in some embodiments, the subject may be in the prone position on a surgical table 200 and the spine may be accessed laterally. While in the prone position, a first incision 202 may be made at about the location of the iliac crest 28 in order to gain access to the iliac crest 28. A second incision 204 may be made proximate the first incision 202 and positioned caudally to the first incision 202. The first and second incision 202, 204 may be separated by a distance. In some embodiments, the distance may be about 2-6 inches, however, other distances may also be used without departing from aspects of the disclosure. A cannula 206 can optionally be inserted through the first and second incision 202, 204 to provide a subcutaneous channel between the first and second incision 202, 204. A free end of the pulley 112 opposite the engagement member 108 can be inserted within the cannula 206 from the first incision 202 and out of the second incision 204 so that the free end of the pulley 112 is held by a medical professional. The cannula 206 can be later removed by backing out the cannula 206 over the free end of the pulley 112. In other embodiments, forceps or other medical instruments can be used to pass and/or pull through the pulley 112 from the first incision 202 to the second incision 204.

The pulley 112 can be pulled until the engagement member 108 is positioned proximate the iliac crest 28. The medical professional can then manipulate and/or position the engagement member 108 until it is in a desired position about the iliac crest 28. The tensioning mechanism 114 can be operatively associated with the pulley 112. The pulley 112 can then be pulled by a medical professional until a desired position of the iliac crest 28 is achieved. The medical professional can then secure the pulley 112 to a bar 210 of a surgical table 200 to hold and/or maintain the position of the iliac crest 28. The tensioning mechanism 114 can be actuated to provide further securement of the pulley 112 in this position and/or can be actuated further to provide traction and/or tension on the pulley 112 such that additional movement of the iliac crest 28 may achieved. Once the iliac crest 28 is in a desired position, the desired surgical technique can be performed, e.g., a lateral surgical technique.

It is to be understood that embodiments described herein can be used to displace the iliac crest to any number of desired spinal surgical techniques, and while shown and described relative to a spinal procedure, the iliac crest and displacement device can be used in other medical applications or procedures where it may be desirable to displace the iliac crest.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The terminology used herein is for the purpose of describing particular embodiments only and is not intended to be limiting of the disclosure. As used herein, the terms “first,” “second,” and the like, do not denote any order, quantity, or importance, but rather are used to distinguish one element from another, and the terms “a” and “an” herein do not denote a limitation of quantity, but rather denote the presence of at least one of the referenced item. It will be further understood that the terms “comprises” and/or comprising,” when used in this specification, specify the presence of stated features, integers, steps, operations, elements, and/or components, but do not preclude the presence or addition of one or more other features, integers, steps, operations, elements, components, and/or groups. As used herein, “substantially” refers to largely, for the most part, entirely specified or any slight deviation which provides the same technical benefits of the disclosure.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed is:

1. An iliac crest displacement device comprising:
an engagement member for engaging an iliac crest of a subject; and
a pulley assembly for causing the engagement member to move the iliac crest inferiorly, thereby displacing the iliac crest relative to a rib cage of the subject.

2. The iliac crest displacement device of claim 1, wherein the engagement member includes a hook, saddle, grasp, anchor, latch, or clamp.

3. The iliac crest displacement device of claim 1, further comprising:
a pin configured to be fixed to the iliac crest,
wherein the engagement member is configured to couple with the pin.

4. The iliac crest displacement device of claim 3, wherein the pin includes an anchor, a traction pin, or a fixation element.

5. The iliac crest displacement device of claim 1, wherein the pulley assembly includes a pulley and a tensioning mechanism.

6. The iliac crest displacement device of claim 5, wherein a first end of the pulley is coupled to the engagement member.

7. The iliac crest displacement device of claim 6, wherein the tensioning mechanism is configured to provide tension to the pulley thereby maintaining a position of the pulley.

8. The iliac crest displacement device of claim 6, wherein a second end of the pulley, opposite the first end, is configured to be grasped by a medical professional to move the iliac crest inferiorly.

9. The iliac crest displacement device of claim 6, wherein s second end of the pulley, opposite the first end, is configured to be fixed to a surgical table or bar.

10. The iliac crest displacement device of claim 5, wherein the tensioning mechanism includes a ratchet tensioner.

11. The iliac crest displacement device of claim 5, wherein at least a portion of the pulley is configured to be positioned subcutaneously about the subject, and wherein a remainder portion of the pulley and the tensioning member is configured to be positioned external to the subject.

12. The iliac crest displacement device of claim 5, wherein the pulley includes a tie, cord, tether, strap, line, band, rope, or wire.

13. The iliac crest displacement device of claim 5, wherein at least a portion of the pulley is positioned subcutaneously extending from the engagement member in the inferior direction subcutaneously.

14. The iliac crest displacement device of claim 13, wherein a remainder of the pulley and the tensioning mechanism are positioned external to the patient.

15. A method for accessing a surgical target site of a subject, the method comprising:
accessing an iliac crest of a subject;
providing an iliac crest displacement device, the iliac crest displacement device including:
an engagement member for engaging the iliac crest of the subject; and
a pulley assembly for causing the engagement member to move the iliac crest inferiorly;
engaging the iliac crest with the engagement member;
using the pulley assembly to cause the iliac crest to move inferiorly thereby displacing a pelvis of the subject relative to a rib cage of the subject; and
providing tension to the pulley to hold the position of the iliac crest in the displaced position.
